(19) United States
(12) Patent Application Publication
(10) Pub. No.: US 2019/0374461 A1
(43) Pub. Date: Dec. 12, 2019

(54) Treatment apparatus and mechanisms for an infected nail

(71) Applicant: Todd Alan Weinfield, Redwood City, CA (US)

(72) Inventor: Todd Alan Weinfield, Redwood City, CA (US)

(21) Appl. No.: 15/998,012

(22) Filed: Jun. 11, 2018

(51) Int. Cl. A61K 9/00; A61K 47/18; A61F 7/02; A61P 31/04

(52) U.S. Cl. CPC A61K 9/0014; A61K 47/18; A61F 2007/0037; A61P 31/04; A61F 7/02

(57) ABSTRACT

A method of treating nail infections that uses nail protein removing compositions to reduce cell to cell cohesion; antimicrobial composition to reduce surface tension, inhibit spores, and attack infection cell membranes; an enclosure capable of heating the antimicrobial composition above nail temperature and capable of dispensing antimicrobial composition to infected nails.

### CROSS REFERENCE TO OTHER APPLICATIONS

This application claims the benefit of provisional patent application 62/604,654 filed on Jul. 15, 2017 entitled Nail Infection Treatment Apparatus, Methods, Mechanisms, Modes of Action and Processes.

### FEDERALLY SPONSORED RESEARCH OR DEVELOPMENT

None.

### BACKGROUND OF THE INVENTION

### Field of the Invention

The teachings provided herein are directed to methods and compositions that facilitate the delivery of therapeutic composition(s) through the nail of a subject having a nail infection.

### Prior Art

The development of a cure for nail infections has historically focused on solving only one problem—delivering known antimicrobial medication (developed to treat skin infections) through the infected nail to treat the condition. Approximately 10% of the U.S. population under the age of 70 is infected with the condition, but that figure increases to 48% for those over 70. The infection is especially risky for diabetics. If uncontrolled, diabetics risk limb amputation. One third of the world's nearly 400 million diabetics' nails are infected.

Current approaches have yielded only marginal results with cure rates for the best treatments under 40% only for surface or mild infections not involving the nail bed. Clearly, a more effective approach is needed.

The nail provides a seemingly impenetrable membrane protecting the infection from outside elements as Quintanar-Guerrero et al from Universidad Nacional Autonoma de Mexico have shown n in their paper The effect of keratolytic agents on the permeability of three imidazole antimycrobial drugs through the human nail (Drug Dev Ind Pharm, Jul. 1, 1998; 24(7): 685-90).

Quintanar-Guerrero et al also found that keratolytic substances such as papain. and salicylic acid used in combination did enhance the permeability of the antimycrobial. Patents with methods, formulae, and apparatus to increase the permeability of the antimycrobial through the nail into the nail bed dominate this art See, for example, the following U.S. patents and patent application publications: U.S. Pat. Nos. 6,821,508; 6,921,529; 5,795,314; 6,727,401; 6,465,709.

The prior art also includes many topical therapies for the treatment of nail infections. Bohn in U.S. Pat. No. 4,957,730, describes a nail varnish containing a water-insoluble film-forming substance and antimycrobial compound. Ferro in U.S. Pat. No. 5,120,530, teaches an antimycrobial nail varnish containing amorolfine in quaternary ammonium acrylic copolymer. Bohn in U.S. Pat. No. 5,264,206, describes a nail lacquer with antimycrobial activity which contains an antimycrobial agent and water-insoluble film formers including polyvinyl acetate.

Wohlrab in U.S. Pat. No. 5,346,692, describes a nail lacquer for treating nail infections, comprised of a film-forming agent, an antimycrobialally active substance, and urea. Nimni in U.S. Pat. No. 5,487,776, describes a nail lacquer composition which forms a water permeable film containing griseofulvin. Chaudhuri in U.S. Pat. No. 6,143,794, describes a topical formulation for the treatment of nail infections that includes an antimicrobial, solvent, gelling agent, adhesion-promoting agent, a film-forming agent, a surfactant, and optionally a keratolytic agent.

Knowles in U.S. Pat. No. 5,652,256, describes the use of methyl acetate as a penetration enhancing compound in combination with naftifine or sulconazole and naftifine as a topical gel for treatment of nail infections. Sorenson in U.S. Pat. No. 5,972,317, discloses that a proteolytic enzyme such as papain that is delivered by pads soaked in the enzyme solution and produces a more permeable nail. Sun in U.S. Pat. No. 6,231,875, teaches acidified compositions of antimicrobials to enhance transport across nails and skin. Reeves in U.S. Pat. No. 6,391,879 describes the combination of an antimicrobial agent in an anhydrous blend of polyglycol and DMSO.

Birnbaum in U.S. Pat. No. 7,135,194, discloses a subunguicide with an antimicrobial agent administered between the hyponychium and the nail plate. Winckle in U.S. Pat. No. 8,039,494 teaches a composition containing a triazole antimicrobial pharmaceutically active agent, a solvent, and a wetting agent in a composition which does not form a solid film when applied to the surface of a nail.

Bailey in U.S. Pat. No. 8,979,820 teaches a sequence of steps how to improve the appearance of infected nails that includes soaking them in camphor and boric acid. Baker in US 2007/0054834 describes using quaternary ammonium halides to treat infectious conditions on skin. Mallard et al in US 2012/0328543 teaches using surfactants to facilitate the delivery of antimicrobial agents into the infected nail.

Other U.S. patents that teach combination antimicrobial products include, for example: U.S. Pat. No. 4,636,520 (combination of imidazole and pyrrolnitrin); U.S. Pat. No. 5,002,938 (gel combination of imidazole and 17-ester corticosteroid anti inflammatory agent); U.S. Pat. No. 5,110,809 (antimicrobial gel plus steroid); U.S. Pat. No. 5,219,877 (gel product with imidazole antimicrobial optionally with steroidal anti-inflammatory in a vehicle system that includes lauryl alcohol); U.S. Pat. No. 5,391,367 (aqueous alcoholic gel with tioconazole); U.S. Pat. No. 5,464,610 (salicylic acid plaster); and U.S. Pat. No. 5,696,105 (mometasone furoate).

Existing or protocols in development for treating nail infections fit into one of four categories: 1) oral or systemic; 2) topical; 3) light, electromagnetic or gas plasma; 4) iontophoresis or electrophoresis.

1) Oral treatments include Griseofulvin, Ketoconazole, Lamisil, Sporanox, Posaconazole, and Albaconazole (a typical 12 month course costs between $1,350 and $2,700). Lamisil has been the most effective with a mycological cure rate of 46%. The limited success of Lamisil in the market is attributed to its potential for toxic side effects, high relapse rate, long treatment times, marginal cure rates, and the tendency of the infection to inhabit portions of the nail which are not vascularized. Posaconazole and Albaconazole post similar results but are prone to fewer side effects than Lamisil.

2) Topical treatments include: amorolfine, ciclopirox, efinaconazole, tavaborole, and luliconazole. Amorolfine (Galderma) does not have FDA approval is not available in the United States, however, has shown cure rates of 40 to 55% for mild nail infections without nail matrix involvement, however, it is not effective against severe nail infections involving the nail bed. A typical course of amorolfine is 12 months and costs $1,080. Ciclopirox is the only FDA approved medication for the condition and has cure rate for the 8% strength of about 8.5%.

Valeant's efinaconazole demonstrated cure rates of 17.8% and 15.2% only for mild infections not involving the nail bed. Analysts estimate peak annual sales of efinaconazole at $200 million. Anacor's Tavaborole (boron-based) achieved complete cure in 6% of the 600 patients in the study. Other topical treatments of less interest include: Cindacin (DSMO and tolnaftate from Pedicis Research); Emtrix, Nalox, and Kerasal Nail (urea, propylene glycol and lactic acid from Paladin Labs) are used to treat symptoms of nail infections or psoriasis including discolored or brittle nails, but not treat the condition itself.

3) Light, electromagnetic and gas plasma Companies such as Keraderm, Patholase, Nomir, and Devicefarm have developed light energy and gas plasma technology to treat infections taking advantage of UV, laser light and chlorine gas plasma's ability to inhibit growth of the infection in vitro. The build up of keratin debris under the nail plate prevents light and plasma from penetrating the nail. The best published effective rate for light energy is Patholase with a 10% cure rate (placebo is 6%) administered during an office visit for $900-$1,200 per application.

Devicefarm gas plasma ($1,500 to $2,000 for three office visits) has not published the results from their clinical trials despite evidence that skin infections require more than three treatments to cure, the concentration of chlorine necessary to effectively treat dermatophyties is toxic to human skin and the plasma fails to completely penetrate even half the thickness of a normal nail.

Dusa Pharmaceuticals patented photodynamic therapy in which a therapeutic agent is applied to the nail several days prior to exposing the nail to light that causes an activation reaction. The published data for photodynamic therapy since 2008 suggests it improves the appearance of infected nails but is unable to penetrate deep enough to effectively treat the infection.

4) Iontophoresis, electrophoresis and implant Startup NB Therapeutics (Nitric Biotherapeutics) developed an iontophoretic process that transports terbinafine hydrochloride (Lamisil) through the nail. Unfortunately, the results of the clinical trials were unable to convince investors to continue pursuing this approach so the company failed. Lamisil implant startup Hallux (formerly Talima) has only started recruiting for clinical trials.

None of these methods have been shown to be consistently clinically effective in treating nail infections. For this reason, a successful, safe, and noninvasive topical treatment for nail infections is a long-felt and unsolved need which is well-understood and accepted by those skilled in the art.

Accordingly, those skilled in the art of treating nail infections, and the patients suffering such nail infections, will appreciate a successful, safe, and non-invasive topical treatment for nail infections. The present teachings provide such a method of treatment that (i) is topical and safe; (ii) does not require oral or systemic administration of drugs; (iii) is safer for patients that may be intolerant to systemic drug delivery; (iv) is several times faster than existing topical treatments; (v) does not require removal of the nail; and, as such, (vi) does not require the patient to do without the presence of a nail for the year or so required to grow a new nail. These are examples of the advantages that will be realized in the art by the teachings provided herein.

The treatment of an infected nail requires administering an mechanism to a subject having an infected nail. The terms “administration” or “administering” refer to a method of incorporating a mode of action into the cells or tissues of a subject, either in vivo or ex vivo to diagnose, prevent, treat, or ameliorate a symptom of a disease. In one example, a mechanism can be administered directly to the affected tissue of a subject.

In another example, a mechanism can be administered to a subject by combining the mechanism with cell tissue from the subject ex vivo for purposes that include, but are not limited to, assays for determining utility and efficacy of a mode of action. When the mechanism is incorporated on the subject in combination with one or active mechanisms, the terms “administration” or “administering” can include sequential or incorporation concurrent incorporation of the mechanism with the other mechanisms such as, for example, any mechanism described above.

A pharmaceutical mechanism of the invention is formulated to be compatible with its intended route of administration. An “effective amount” of a mechanism of the invention can be used to describe a therapeutically effective amount or a prophylactically effective amount. An effective amount can also be an amount that ameliorates the symptoms of a disease. A “therapeutically effective amount” refers to an amount that is effective at the dosages and periods of time necessary to achieve a desired therapeutic result and may also refer to an amount of active mechanism, prodrug or pharmaceutical mechanism that elicits any biological or medicinal response in a tissue, system, or subject that is sought by a researcher, veterinarian, medical doctor or other clinician that may be part of a treatment plan leading to a desired effect.

### BRIEF SUMMARY OF THE INVENTION

Whereas treating infected nails with protein disrupting and antimicrobial mechanisms is known in this art, new to this art and discovered unexpectedly is the selection and application of protein disruptive and antimicrobially active mechanisms in warm water that optimizes cure rates and convenience but minimizes treatment time and patient risk. To that end and new to this art is the scientific breakdown and understanding of the individual challenges in treating nail infections which, when assembled into a single targeted approach, effectively treats the condition in vivo regardless of severity, etiology or age of the infection. Below is that breakdown.

A first aspect for these embodiments is nail hydrophilicity. Known in this art is that nail keratin is hydrophilic (absorbs water). New to this art is the discovery that nail keratin will also absorb larger molecules (>18 g/mol) and deliver them with their modes of action intact through the nail to effectively treat infections.

A second aspect for these embodiments is energy from heat. Known in this art is that energy (heat) is required for water molecules to penetrate nail keratin to reach the infection (why nails become soft in warm water and harden in cold water). New to this art is the discovery that heat energy drives higher molecular weight antimycrobially active mechanisms (>18 g/mol) through nail keratin to effectively treat infections at temperatures greater than recommended without denaturing them.

A third aspect for these embodiments is nail thickness. Known in this art is that keratolytic mechanisms applied to nail keratin reduce the thickness of the nail and keratin debris (infection waste product) and enhance the delivery of antimycrobially active mechanisms. New to this art is the application of keratolytic mechanisms that allow antimicrobials with more potent mechanisms to penetrate the nail to effectively treat the infection.

A fourth aspect for these embodiments is electrostatic repulsion. Known in this art is the electrostatic build up between the antimycrobial and the nail that prevents penetration. New to this art and discovered unexpectedly is that cationic antimicrobial surfactant mechanisms facilitate this delivery in addition to adding modes of action to treat the infection where non-ionic, anionic, amphoteric, sufactatergent and emulsifiers are less effective.

A fifth aspect for these embodiments are the potency of the antimicrobial mechanisms. Known in this art to treat nail infections is the delivery of established antimicrobial mechanisms to the nail. Traditional antimycrobials usually exhibit no more than one or two modes of action to treat the condition which allows the infection to build up resistance. New to this art is the delivery of more powerful antimicrobial mechanisms (with potency inferred from antibacterial studies) that inhibits infection resistance by disabling many cellular functions simultaneously.

A sixth aspect for these embodiments is molecular weight and orientation. Known in this art is that smaller molecular weight antimycrobials are able to accumulate in higher concentrations under the nail because of their smaller size. New to this art is the discovery of the delivery of higher molecular weight antimycrobials with more powerful mechanisms accumulate under the nail in high enough concentrations to effectively treat the infection despite their larger size. Driving this delivery is the discovery of antimycrobials with mechanisms with molecular orientation that align with the nail plate structure to allow penetration.

Therapeutic foot baths that relieve the symptoms of foot conditions are well known in this art. New to this art is the combination of foot baths with active antimycrobial mechanisms to treat nail infections. The benefits of this combination include minimizing the amount of medication necessary, isolating the infection, minimizing exposure to non-infected tissue and lowering treatment costs; making the treatment portable; providing a practical and economical home treatment with a pre-heated option combined with mechanisms to treat the infection instead of its symptoms.

### DRAWINGS

Table A Embodiment 1—Heating Soaking Pan (FIGS. 1-3)

FIG. 1 Isometric View

FIG. 2 Cross Section View

FIG. 3 Bottom View

Table B Embodiment 2—Heated Spray Device (FIGS. 4-7)

FIG. 4 Isometric View

FIG. 5 Isometric View

FIG. 6 Cross Section View

FIG. 7 Bottom View

Table C Embodiment 3 Heated Agitation Device (FIGS. 8-11)

FIG. 8 Isometric View

FIG. 9 Isometric View

FIG. 10 Cross Section View

FIG. 11 Bottom View

Table D Embodiment 4—Microwave Heated Soaking Pan (FIGS. 12-13)

FIG. 12 Isometric View

FIG. 13 Cross Section View

### DETAILED DESCRIPTION OF THE INVENTION

The following description of the invention will typically be with reference to embodiments and methods. It is to be appreciated that there is no intention to limit the invention to the specifically disclosed embodiments and methods but that the invention may be practiced using other features, elements, methods and embodiments not to limit its scope, which is defined by the claims. Those of ordinary skill in the art will recognize a variety of equivalent variations on the description that follows.

### Apparatus

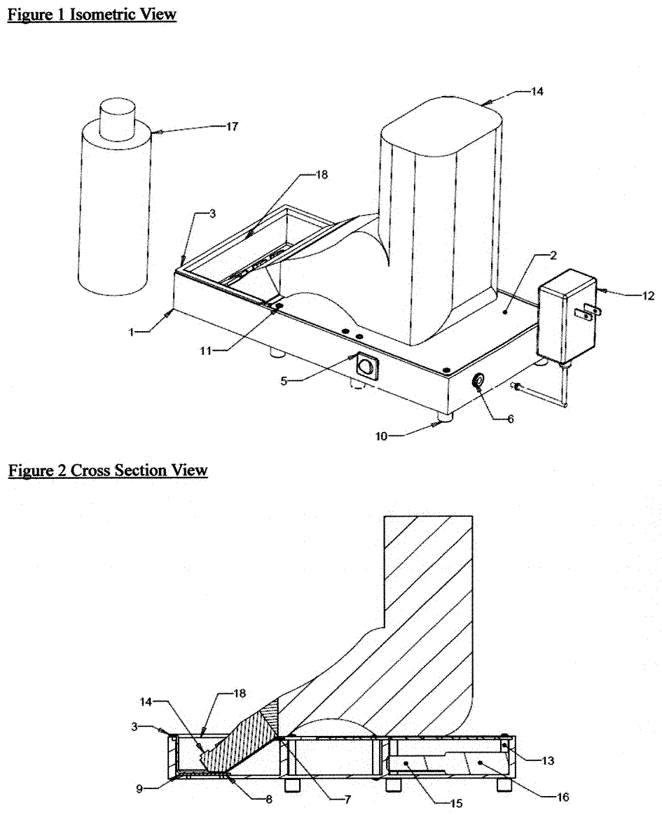

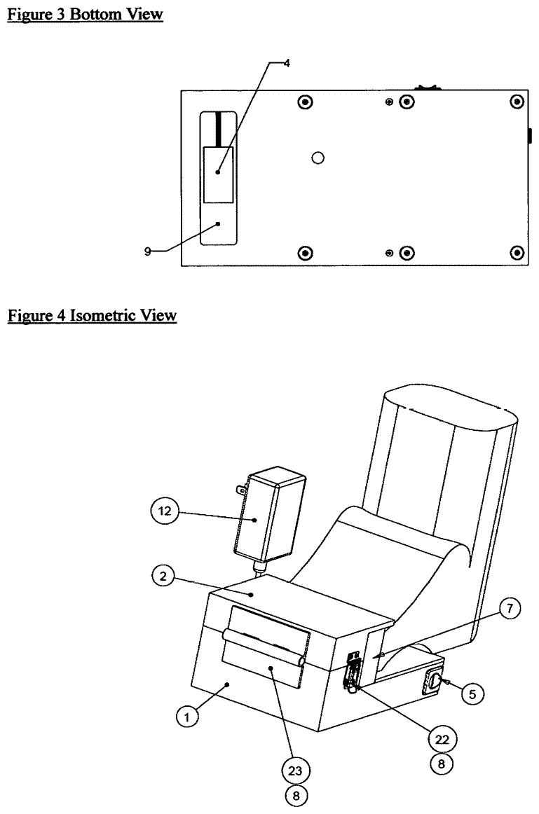

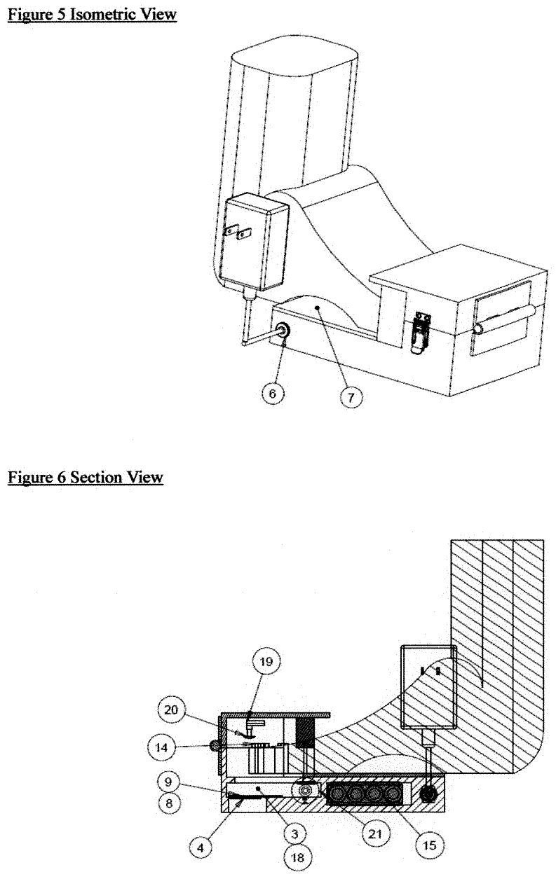

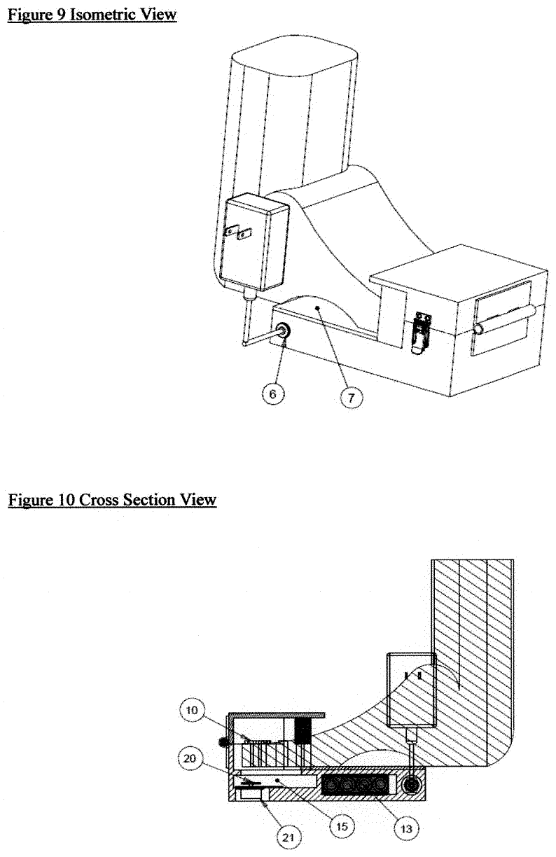

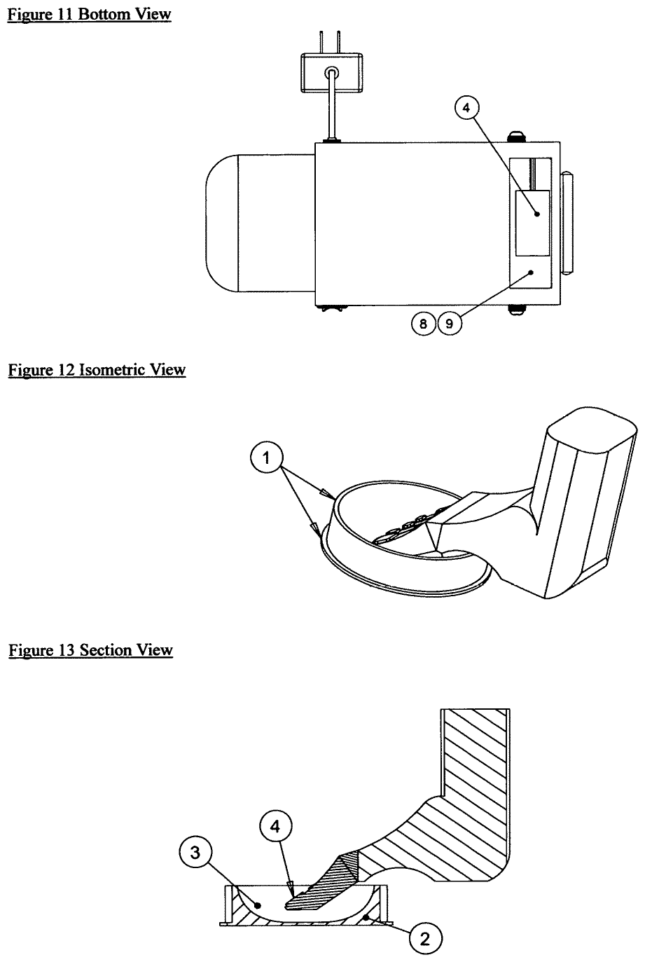

Accordingly, Tables A, B, C and D and FIGS. 1-13 provide a detailed description of the components used in the heated soaking device apparatus.

### Table A Embodiment 1—Heated Soaking Pan (FIGS. 1-3)

Base 1 can be made of any rigid corrosion resistant material including aluminum or ABS plastic and functions as the support structure for infected nail 14 and soaking pan 3. Soaking pan 3 is made from any thermally conductive corrosion resistant metal such as aluminum or copper and creates the volume that encloses treatment solution 18, heater 4, and the infected nails 14. Heater 4 is typically an off the shelf resistance type heater, and is made from thermally conductive metal (copper, aluminum) wires embedded in kapton or silicone. Base cover 2 can be made from any corrosion resistant material including aluminum or ABS plastic and serves to cover the electrical components inside base 1 and to support infected nail 14.

Treatment solution 18 is made from off the shelf components and mixed to the proper concentration to treat infections. Treatment solution container 17 is purchased off the shelf and is made from a variety of materials, most commonly the same inert plastic material as the original packaging for treatment solution 18 or HDPE. Power switch 5 is screwed into base 1 and turns the device on or off Power connector 6 is screwed into base 1 and relays the power from power supply 12 (plugged into a wall outlet) through power switch 5 to battery charger 16 to heater 4. Battery charger 16 is mounted inside base 1 and uses electricity from power supply 12 to recharge batteries 15 for portable use.

High temperature adhesive 8 is typically silicone based and is purchased off the shelf to secure heater 4 to base 1. Insulation 7 is purchased off the shelf and is made from a variety of inert materials including buna nitrile and neoprene and insulates the electrical components inside base 1 from treatment solution 18. Corrosive resistant screws 11 (aluminum or stainless steel) secure rubber bumpers 10 to base 1 through standoffs 13. Rubber bumpers 10 create space under the device for heater 4 and also electrically insulate the device from ground.

Treatment solution 18 is poured from treatment solution container 17 into soaking pan 3 to cover infected nail 14 and heated using heater 4 and heater plate 9. Infected nail 14 is soaked for 15 minutes once daily for three to four months at 45° C. max.

### Table B Embodiment 2—Heated Spray Device (FIGS. 4-7)

Base 1 can be made of any rigid corrosion resistant material including aluminum or ABS plastic and functions as the support structure for infected nail 14, soaking pan/reservoir 3 and base cover 2. Soaking pan/reservoir 3 is a cavity inside base 1 and creates the volume that encloses treatment solution 18, heater plate 9 and the infected nails 14. Heater 4 is an off the shelf resistance type heater and can be made from a nickel-chromium alloy embedded in kapton or silicone and transfers heat through heater plate 9 to treatment solution 18.

Heater plate 9 can be made of any thermally conductive material such as aluminum or copper and transfers heat from heater 4 to treatment solution 18 and seals soaking pan/reservior 3 using high temperature adhesive 8. Base cover 2 can be made from any corrosion resistant material including aluminum or ABS plastic and serves to cover infected nails 14 and provide support for insulation 7. Insulation 7 is purchased off the shelf and is made from a variety of inert materials including buna nitrile and neoprene and provides a liquid seal to keep treatment solution 18 inside base 1 and base 2.

Treatment solution 18 is made from off the shelf antimicrobial components and mixed to the proper concentration to treat infections. Power switch 5 is a purchased part and is screwed into base 1 and controls the electrical connection between heater 4, rechargeable batteries 15, power supply 12 and pump 21 to heat and spray treatment solution 18 onto infected nail(s) 14. Power connector 6 is screwed into base 1 and relays the power from power supply 12 (plugged into a wall outlet) through power switch 5 to rechargeable batteries 15 to heater 4. Rechargeable batteries 15 are purchased off the shelf and used to operate the device away from a wall outlet. Power supply 12 is a purchased part and converts electricity from a wall outlet so it's useable by heater 4 and pump 21.

High temperature adhesive 8 is usually silicone based and is purchased off the shelf to secure heater 4 to base 1 and also secures hinge 23 and latch 22 to base 1 and base 2. Hinge 23 is a purchased part and can be made from aluminum, steel, or ABS plastic and allows base 2 to rotate open to insert infected nail(s) 14 and treatment solution 18. Latch 22 is a purchased component made from metal or plastic and secures base 1 and base 2 together.

Treatment solution 18 is poured into soaking pan/reservoir 3 where pump 21 pumps it through tubing 19 and spray nozzle 20 onto infected nail(s) 14. Pump 21, tubing 19, and spray nozzle 20 are all purchased components.

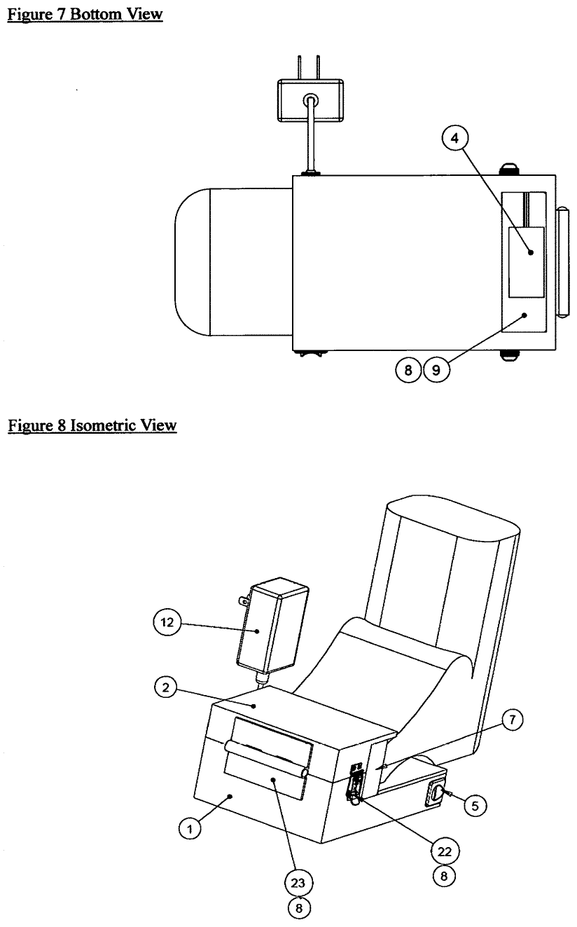

### Table C Embodiment 3—Heated Agitation Device (FIGS. 8-11)

Base 1 can be made of any rigid corrosion resistant material including aluminum or ABS plastic and functions as the support structure for infected nail 14, soaking pan/reservoir 3 and base cover 2. Soaking pan/reservoir 3 is a cavity inside base 1 and creates the volume that encloses treatment solution 18, heater plate 9 and the infected nails 14. Heater 4 is an off the shelf resistance type heater and can be made from a nickel-chromium alloy embedded in kapton or silicone and transfers heat through heater plate 9 to treatment solution 18.

Heater plate 9 can be made of any thermally conductive material such as aluminum or copper and transfers heat from heater 4 to treatment solution 18 and seals soaking pan/reservior 3 using high temperature adhesive 8. Base cover 2 can be made from any corrosion resistant material including aluminum or ABS plastic and serves to cover infected nails 14 and provide support for insulation 7. Insulation 7 is purchased off the shelf and is made from a variety of inert materials including buna nitrile and neoprene and provides a liquid seal to keep treatment solution 18 inside base 1 and base 2.

Treatment solution 18 is made from off the shelf antimicrobial components and mixed to the proper concentration to treat infections. Power switch 5 is a purchased part and is screwed into base 1 and controls the electrical connection between heater 4, rechargeable batteries 15, power supply 12 and motor 21 to heat and agitate treatment solution 18 onto infected nail(s) 14 using impeller 20. Power connector 6 is screwed into base 1 and relays the power from power supply 12 (plugged into a wall outlet) through power switch 5 to rechargeable batteries 15 to heater 4. Rechargeable batteries 15 are purchased off the shelf and used to operate the device away from a wall outlet. Power supply 12 is a purchased part and converts electricity from a wall outlet so it's useable by heater 4 and motor 21.

High temperature adhesive 8 is usually silicone based and is purchased off the shelf to secure heater 4 to base 1 and also secures hinge 23 and latch 22 to base 1 and base 2. Hinge 23 is a purchased part and can be made from aluminum, steel, or ABS plastic and allows base 2 to rotate open to insert infected nail(s) 14 and treatment solution 18. Latch 22 is a purchased component made from metal or plastic and secures base 1 and base 2 together.

Treatment solution 18 is poured into soaking pan/reservoir 3 where motor 21 and impeller 20 agitate it onto infected nail(s) 14. Motor 21 and impeller 20 are purchased parts. Motor 21 rotates impeller 20.

### Table D Embodiment 4—Microwave Heated Soaking Pan (FIGS. 12-13)

Base and cover 1 are purchased components and are made of a microwaveable plastic such as polypropylene. Treatment solution 3 is poured into the base open volume and the cover seals insulation 2 inside the base closed volume. Base and cover 1, insulation 2 and treatment solution 3 is placed in the microwave and warmed until treatment solution 3 is a maximum temperature of 45° C. Infected nail(s) 4 are soaked in warm treatment solution 3. Insulation 2 is purchased off the shelf and is made from a variety of inert materials including buna nitrile, neoprene and water and keeps treatment solution 3 warm while infected nail(s) 4 are soaking.

### Mechanisms

Protein removing mechanisms that disrupt, remove protein and reduce cell to cell cohesion are typically used in treating acne and warts on skin. Compositions with protein removing mechanisms are well known and available from commercial laboratories such as Santa Cruz Biotechnology (SC) and Sigma Aldrich (SA) under such part numbers as: SC-215227; 229790; 215933; 203371; 296681; 203374; 29114; 250284; 209002; 210706; 257106; 391055; 214588; and SA-05670; P4762; 777161; P-7545; P5380; R7632 or combinations thereof.

Protein removing compositions in safe concentrations range from 30% to 50% (applied in combination or individually) may be applied to the infected nail(s) separately once per day for up to 90 days or included with the treatment solution and heated for soaking the infected nail(s).

Antimicrobial cationic surfactant mechanisms that reduce surface tension, inhibit spore outgrowth, disrupt cytoplasmic membranes, degrade proteins and nucleic acids and lysis cell walls are typically used as surface disinfectants. Compositions with antimicrobial cationic surfactant mechanisms are well known and available from commercial laboratories such as: Makon® NF-5 (Stepan Company, Northfield, Ill.); Arquad@ 2HT-75 (Akzo Chemicals Inc., Chicago, Ill.); Kemamine BQ-9742C (Witco Chemical Corp., Memphis, Tenn.) Kemamine Q-9702C (Witco Chemical Corp.), Accosoft 750 (Stepan Co. Northfield, Ill.) and Accosoft 501 (Stepan Company).

Antimicrobial cationic surfactant compositions in safe concentrations less than 0.3% are heated above nail temperature and penetrate the infected nail with water.

Nitrogenous carbon antimicrobial compositions attach to phospholipids in cytoplasmic membranes and cause an irreversible loss of essential cellular components. These mechanisms also bind to DNA and other nucleic acids and precipitates them from aqueous solution thereby damaging or inactivating DNA.

Compositions with nitrogenous carbon mechanisms are well known and are typically used as public water treatments and surface disinfectants. These antimycrobials include but are not limited to and are commercially available: Lonza (88865); Aventis Pharma (Brolene); Typharm (Golden Eye); Merck (Aurigoutte); Pierre Fabre (Cyteal); and Betadine (Betasept).

Nitrogenous carbon compositions in safe concentrations less than 0.3% are heated above nail temperature and penetrate infected nail(s) with water.

One or more antimicrobial cationic surfactant (concentration in water less than 0.3%) and/or one or more nitrogenous carbon antimicrobial (concentration in water less than 0.3%) are mixed with water and together are absorbed into the infected nail(s). Protein removing compositions may be applied separately to infected nail(s) (concentration less than 30%) or included with the antimicrobial cationic surfactant and the nitrogenous carbon composition with water (concentration less than 50%).

The mixture is heated above nail temperature (usually usually 5°-10° C. less than body temperature of 37° C. and less than the FDA maximum of 45° C.). Heat energy helps the mixture overcome the infected nail's natural resistance to absorption (why nails soften in warm water and harden in cold water).

Embodiments may include one or more protein removing composition, one or more antimicrobial cationic surfactant composition and water heated above nail temperature. Other embodiments may include one or more protein removing composition, one or more nitrogenous carbon composition and water heated above nail temperature. Further embodiments may include one or more protein removing composition, one or more antimicrobial cationic surfactant composition, one or more nitrogenous carbon composition and water heated above nail temperature.

Safe therapeutically effective concentrations of keratolytic ingredients for all embodiments is less than 50%; for antimicrobial cationic surfactant composition is less than 0.3%; and for nitrogenous carbon composition less than 0.3%. Temperature range for all embodiments is above nail temperature (usually 5°-10° C. less than body temperature of 37° C. and less than the FDA maximum of 45° C.).

Treatment duration depends on the severity of the infection and ranges from soaking infected nails in warm treatment solution from 5 to 15 minutes once per day for 20 to 120 days.

The treatment of an infected nail requires administering an agent to a subject having an infected nail. The terms “administration” or “administering” refer to a method of incorporating a composition into the cells or tissues of a subject, either in vivo or ex vivo to diagnose, prevent, treat, or ameliorate a symptom of a disease. In one example, a compound can be administered directly to the affected tissue of a subject. In another example, a compound can be administered to a subject by combining the compound with cell tissue from the subject ex vivo for purposes that include, but are not limited to, assays for determining utility and efficacy of a composition. When the compound is incorporated on the subject in combination with one or active agents, the terms “administration” or “administering” can include sequential or incorporation concurrent incorporation of the compound with the other agents such as, for example, any agent described above. A pharmaceutical composition of the embodiment is formulated to be compatible with its intended route of administration.

An “effective amount” of a compound of the embodiment can be used to describe a therapeutically effective amount or a prophylactically effective amount. An effective amount can also be an amount that ameliorates the symptoms of a disease. A “therapeutically effective amount” refers to an amount that is effective at the dosages and periods of time necessary to achieve a desired therapeutic result and may also refer to an amount of an active compound, drug or pharmaceutical agent that elicits any biological or medicinal response in a tissue, system, or subject that is sought by a researcher, veterinarian, medical doctor or other clinician that may be part of a treatment plan leading to a desired effect.

The term “treating” refers to the administering one or more therapeutic or prophylactic agents taught herein. A “prophylactically effective amount” refers to an amount that is effective at the dosages and periods of time necessary to achieve a desired prophylactic result such as, preventing or inhibiting the severity of condition. Typically, a prophylactic dose is used in a subject prior to the onset of a disease, or at an early stage of the onset of a disease, to prevent or inhibit onset of the disease or symptoms of the disease. A prophylactically effective amount may be less than, greater than, or equal to a therapeutically effective amount.

In some embodiments, the therapeutically effective amount may need to be administered in an amount sufficient to result in amelioration of one or more symptoms of a disorder, prevention of the advancement of a disorder, or regression of a disorder. In one example, a therapeutically effective amount preferably refers to the amount of a therapeutic mechanism that provides a measurable response of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of a desired action of the mode of action.

### EXAMPLES

### Example 1

A subject having a nail infection diagnosis of a Trichophyton genus was treated using a variety of the methods taught herein. The presence of hyphae in the subject was consistent with Trichophyton rubrum and or Trichophyton mentagrophytes (the diagnosis of mixed infections is difficult to determine accurately). Nails infected with these organisms also produce a chronic type of tinea pedis. Infections involving the interdigital areas can produce erythema, fissuring, and may extend into other portions of the hand or foot.

In this example, the infected nail was periodically trimmed during the 90 day treatment period and pretreated with a 30% protein removing composition once daily. Following pretreatment, the infected nail was soaked in a mixture of 0.10% antimicrobial cationic surfactant composition and 0.15% nitrogenous carbon composition in water and heated above nail temperature (approximately 40° C.) for 10 to 15 minutes daily during the treatment period.

### Example 2

A subject having an nail infection diagnosis of a Trichophyton genus was treated using a variety of the methods taught herein. The presence of hyphae in the subject was consistent with Trichophyton rubrum and or Trichophyton mentagrophytes (the diagnosis of mixed infections is difficult to determine accurately). Nails infected with these organisms also produce a chronic type of tinea pedis. Infections involving the interdigital areas can produce erythema, fissuring, and may extend into other portions of the hand or foot.

In this example, the infected nail was periodically trimmed during the 90 day treatment period. After trimming, the infected nail was soaked in a mixture of 30% protein removing composition, 0.10% antimicrobial cationic surfactant composition and 0.15% nitrogenous carbon composition in water and heated above nail temperature (approximately 40° C.) for 10 to 15 minutes daily during the treatment period.

Examples 1 and 2 show the successful treatment of an infected nail using the teachings described herein. As can be seen from the figures, this treatment eliminates the infection and provides clinical cure.

TABLE A
Embodiment 1 - Heated Soaking Pan (FIGS. 1-3)

| Item | | | | |
|---|---|---|---|---|
| No. | Description | Quantity | Mfg/Supplier | Ref Part No. |
| 1 | Base | 1 | Custom | N/A |
| 2 | Base Cover | 1 | Custom | N/A |
| 3 | Soaking Pan | 1 | Custom | N/A |
| 4 | Heater | 1 | McMaster-Carr | 7945T42 |
| 5 | Power Switch | 1 | Radio Shack | 275-712 |
| 6 | Power Connector | 1 | Radio Shack | 274-1576 |
| 7 | Insulation | A/R | McMaster-Carr | 8635K26 |
| 8 | High Temperature | A/R | McMaster-Carr | 75825A5 |
| | Adhesive | | | |
| 9 | Heater Plate | 1 | Custom | N/A |
| 10 | Rubber Bumper | 6 | Mcmaster-Carr | 9540K724 |
| 11 | Screws | 8 | Mcmaster-Carr | 91735A109 |
| 12 | Power Supply | 1 | Radio Shack | 273-029 |
| 13 | Standoffs | 8 | Mcmaster-Carr | 93330A430 |
| 14 | Infected Nail(s) | 1 | N/A | N/A |
| 15 | Rechargeable | 4 | Eveready | NH15-AA |
| | Batteries | | | |
| 16 | Battery Charger | 1 | Eveready | CHDC-CA |
| 17 | Treatment Solution | 1 | US Plastics | 73036 |
| | Container | | | |
| 18 | Treatment Solution | A/R | Arquad, Lonza | Varies by |
| | | | Sigma Aldrich | Manufacturer |

TABLE B
Embodiment 2 - Heated Spray Device (FIGS. 4-7)

| Item | | | | |
|---|---|---|---|---|
| No. | Description | Quantity | Mfg/Supplier | Ref Part No. |
| 1 | Base | 1 | Custom | N/A |
| 2 | Base Cover | 1 | Custom | N/A |
| 3 | Soaking Pan/ | 1 | Custom | N/A |
| | Reservoir | | | |
| 4 | Heater | 1 | McMaster-Carr | 7945T42 |
| 5 | Power Switch | 1 | Radio Shack | 275-712 |
| 6 | Power Connector | 1 | Radio Shack | 274-1576 |
| 7 | Insulation | A/R | McMaster-Carr | 8635K26 |
| 8 | High Temperature | A/R | McMaster-Carr | 75825A5 |
| | Adhesive | | | |
| 9 | Heater Plate | 1 | Custom | N/A |
| 12 | Power Supply | 1 | Radio Shack | 273-029 |
| 14 | Infected Nail(s) | 1 | N/A | N/A |
| 15 | Rechargeable | 4 | Eveready | NH15-AA |
| | Batteries | | | |
| 18 | Treatment | A/R | Arquad, Lonza | Varies by |
| | Solution | | Sigma Aldrich | Manufacturer |
| 19 | Tubing | A/R | US Plastics | 57270 |
| 20 | Spray Nozzle | 2 | TCP Global | TCPP5500-10 |
| 21 | Submersible | 1 | Edmund (Faller) | 180627 |
| | Pump | | | |
| 22 | Latch | 2 | Southco | 97-30-16--11 |
| 23 | Hinge | 1 | Southco | C6-220 |

TABLE C
Embodiment 3 - Heated Agitation Device (FIGS. 8-11)

| Item | | | | |
|---|---|---|---|---|
| No. | Description | Quantity | Mfg/Supplier | Ref Part No. |
| 1 | Base | 1 | Custom | N/A |
| 2 | Base Cover | 1 | Custom | N/A |
| 4 | Heater | 1 | McMaster-Carr | 7945T42 |
| 5 | Power Switch | 1 | Radio Shack | 275-712 |
| 6 | Power Connector | 1 | Radio Shack | 274-1576 |
| 7 | Insulation | A/R | McMaster-Carr | 8635K26 |
| 8 | High Temperature | A/R | McMaster-Carr | 75825A5 |
| | Adhesive | | | |
| 9 | Heater Plate | 1 | Custom | N/A |
| 10 | Infected Nail(s) | 1 | N/A | N/A |
| 12 | Power Supply | 1 | Radio Shack | 273-029 |
| 13 | Rechargeable | 1 | Eveready | NH15-AA |
| | Batteries | | | |
| 15 | Soaking Pan/ | 1 | Custom | N/A |
| | Reservoir | | | |
| 18 | Treatment Solution | A/R | Arquad, Lonza | Varies by |
| | | | Sigma Aldrich | Manufacturer |
| 20 | Impeller | 1 | NuTone | 68920000 |
| 21 | Motor | 1 | McMaster-Carr | 6331K13 |
| 22 | Latch | 2 | Southco | 97-30-16--11 |
| 23 | Hinge | 1 | Southco | C6-220 |

TABLE D
Embodiment 4 - Microwave Heated Soaking Pan (FIGS. 12-13)

| Item | | | | |
|---|---|---|---|---|
| No. | Description | Quantity | Mfg/Supplier | Ref Part No. |
| 1 | Base and cover | 1 | Bed Bath & | 12356110 |
| | | | Beyond | |
| 2 | Insulation | A/R | medicalproduct | BG1114 |
| | | | sonline.org | |
| 3 | Treatment Solution | A/R | Arquad, Lonza | Varies by |
| | | | Sigma Aldrich | Manufacturer |
| 4 | Infected Nail(s) | 1 | N/A | N/A |

What is claimed is:

1. A method for the treatment of an infection of a nail or nail bed of an individual suffering from said infection, comprising:
a nail protein removing composition that reduces protein cell to cell cohesion;
an antimicrobial composition that reduces surface tension, inhibits spores, and attacks infection cell membranes.

2. The method of claim 1 wherein the antimicrobial composition disrupts proteins in infection cells.

3. The method of claim 1 wherein the antimicrobial composition disrupts nucleic acids in infection cells.

4. The method of claim 1 wherein the antimicrobial composition attaches to infection cell phospholipids.

5. The method of claim 1 wherein the antimicrobial composition is a solution.

6. The method of claim 1 wherein the antimicrobial composition congeals the cytoplasm in the infection cells.

7. The method of claim 1 wherein the components in the antimicrobial composition are dissolved in water.

8. A method for the treatment of an infection of a nail or nail bed of an individual suffering from said infection, comprising:
an enclosure capable of housing at least one digit of a patient with an infected nail or nail bed and;
said enclosure is capable of heating an antimicrobial composition above nail temperature and;
said enclosure is capable of dispensing said antimicrobial composition to said infected nail or nail bed and;
said antimicrobial composition reduces surface tension, inhibits spores and attacks infection cell membranes.

9. The method of claim 8 wherein the enclosure soaks the infected nail or nail bed in an antimicrobial composition.

10. The method of claim 8 wherein the enclosure sprays an antimicrobial composition onto the infected nail or nail bed.

11. The method of claim 8 wherein the enclosure agitates an antimicrobial composition onto the infected nail or nail bed.

12. The method of claim 8 wherein the enclosure uses a pump and spray nozzle to dispense an antimicrobial composition onto an infected nail or nail bed.

13. The method of claim 8 wherein the enclosure uses a motor and impeller to dispense an antimycrobial composition onto an infected nail or nail bed.

14. The method of claim 8 wherein the enclosure uses batteries.

15. A method for the treatment of an infection of a nail or nail bed of an individual suffering from said infection, comprising:
an enclosure capable of housing at least one digit of a patient with an infected nail or nail bed and;
said enclosure is capable of containing an antimicrobial composition;
said enclosure is capable of being externally heated with said antimicrobial composition above nail temperature;
said enclosure is capable of dispensing said antimicrobial composition to said infected nail or nail bed and;
said antimicrobial composition reduces surface tension, inhibits spore outgrowth, and attacks infection cell membranes.

16. The method of claim 12 wherein the enclosure and antimicrobial composition may be heated using a microwave oven.

17. The method of claim 12 wherein the enclosure and antimicrobial composition may be heated using a conventional oven.

18. The method of claim 12 wherein the enclosure and antimicrobial composition may be heated using a stove top.
